(19) United States
(12) Patent Application Publication
(10) Pub. No.: US 2023/0067334 A1
(43) Pub. Date: Mar. 2, 2023

(54) RESTING PUMP

(71) Applicants: Trent Garrett HELLWARTH, Oak Ridge, NC (US); Camden Lee HELLWARTH, Oak Ridge, NC (US)

(72) Inventors: Trent Garrett HELLWARTH, Oak Ridge, NC (US); Camden Lee HELLWARTH, Oak Ridge, NC (US)

(21) Appl. No.: 17/831,113

(22) Filed: Jun. 2, 2022

Related U.S. Application Data
(60) Provisional application No. 63/239,509, filed on Sep. 1, 2021.

(51) Int. Cl. A61M 1/06

(52) U.S. Cl. CPC A61M 1/062; A61M 1/067

(57) ABSTRACT

An apparatus for allowing a user to use a breast pump while resting. The apparatus includes a resting pump mount having a divot sufficiently sized to fit a user's breast. The body substantially holds shape and is at least partially compressible. The apparatus may include comfort features, attached breast pumps, or receptacles to allow for attachment of a breast pump.

### RELATED APPLICATIONS AND CLAIM OF PRIORITY

This patent document claims priority to and is a continuation of U.S. provisional patent application No. 63/239,509, which is fully incorporated into this document by reference.

### BACKGROUND

During the first months of an infant's development, parents often struggle to find time to sleep. This problem is exacerbated during breast feeding or pumping. Lactating individuals can nurse or pump between 8-10 times per day, meaning that they may need to pump up to every 2-3 hours, in some cases, through the night. As a result, they may need wake up multiple times per night to pump. This results in lack of sleep, especially because most breast pumps require the user to be sitting upright and alert while pumping. Disruptions to sleep patterns cause both mental and physical distress.

Therefore, it would be advantageous to have a device that allows a user to rest while breast pumping.

### SUMMARY OF THE DISCLOSURE

Presented is an apparatus for allowing a user to rest while using a breast pump comprising a resting pump mount. The resting pump mount comprises a body having a top, bottom, front, back, first side, and second side; wherein the front side includes a divot sufficiently sized to fit at least one of a user's breasts. The divot is formed by a divot wall, and the body substantially holds shape and is at least partially compressible.

The apparatus may have a divot wall configured to envelop at least the top, front, and bottom of a breast. The apparatus may have a body made of a high density foam. Alternatively, the body may be made of an inflatable material. The apparatus may have a comfort feature made of a soft material, such as velvet, fleece, cotton, polyester, nylon, viscose, or rayon. The comfort feature may be on at least a portion of the divot wall or on at least a portion of the top and/or bottom of the body.

The apparatus may include a breast pump. The breast pump may configured to releasably attached to the resting pump mount or the breast pump is attached to the resting pump mount.

The divot of the apparatus may extend from the first side to the second side. Alternatively, the divot may not contact the first side or the second side.

The apparatus may also include a pump receptacle which may enhance the adaption of a portion of the pump to the resting pump mount. The pump receptacle is located on the divot wall. The pump receptacle may be a pit or a hole in the divot wall.

### BRIEF DESCRIPTION OF THE DRAWINGS

FIG. 1 shows a top front perspective view of an example resting pump mount.

FIG. 2 shows a bottom back perspective view of an example resting pump mount.

FIG. 3 shows a top front perspective view of another example resting pump mount.

### DETAILED DESCRIPTION

In this document, the singular forms “a,” “an,” and “the” include plural references unless the context clearly dictates otherwise. The term “comprising” means “including, but not limited to.” Similarly, the term “comprises” means “includes, and is not limited to.” Unless defined otherwise, all technical and scientific terms used in this document have the same meanings as commonly understood by one of ordinary skill in the art.

In this document, terms that are descriptive of relative position such as “upper” and “lower”, “top” and “bottom”, “horizontal” and “vertical” and the like are intended to indicate relative positions with respect to the components for which those terms are descriptive, and are not intended to be absolute and require that the component remain in that absolute position in all configurations. The term “approximately” includes the recited number and a range of +/−10%,

Except where specifically stated otherwise, numeric descriptors such as “first”, “second”, etc. are not intended to designate a particular order, sequence or position in an overall process or schema, but instead are simply intended to distinguish various items from each other by describing them as a first item, a second item, etc.

This disclosure describes an apparatus configured to allow a user to rest while breast pumping. Further included in this disclosure is a method of using said apparatus.

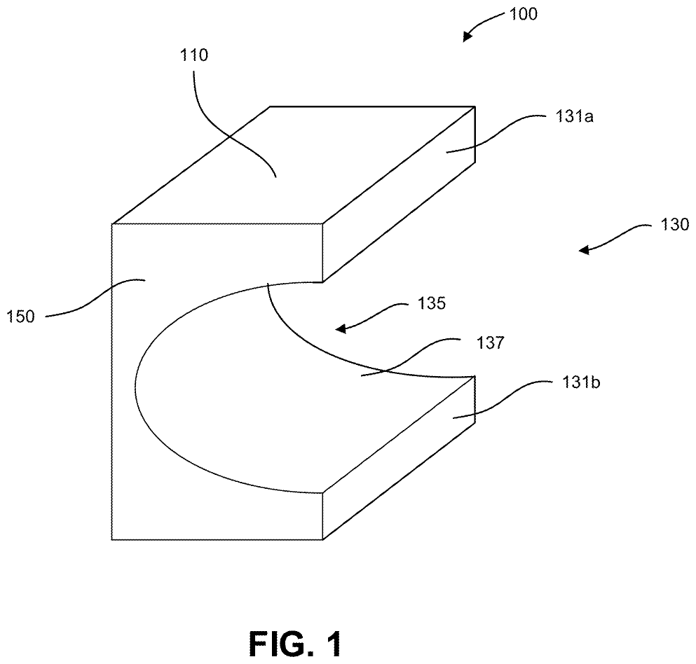

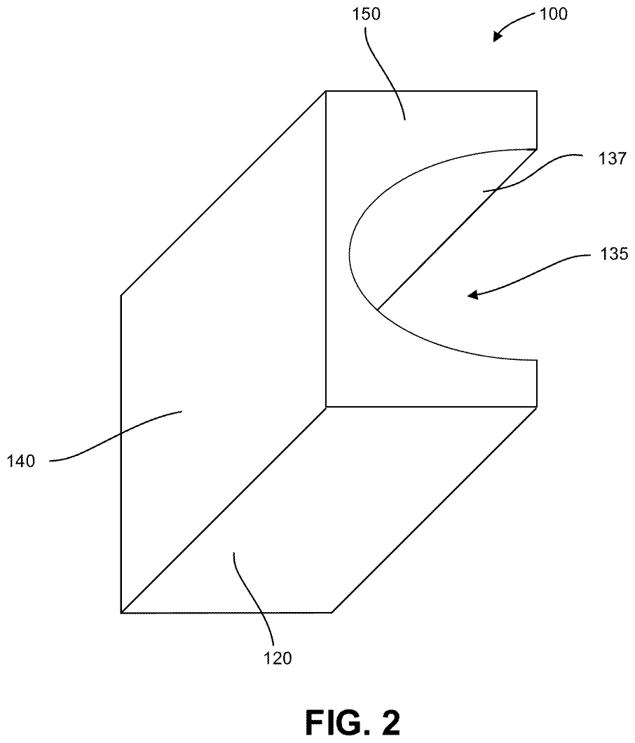

The apparatus of this disclosure may include a resting pump mount 100, as illustrated in FIG. 1. The resting pump mount 100 may have a body with a top 110, a front 130, and two sides 150 (only one side is visible in FIG. 1, the opposite side being a mirror image thereof). As illustrated in FIG. 2, the resting pump mount 100 may have a body with a bottom 120 and a back 140.

In certain embodiments, a lactating individual may use the resting pump mount 100 while sitting. In these embodiments, the bottom 120 of the resting pump mount 100 may rest on the user's lap or legs. The front 130 of the resting pump mount 100 may face the user's torso. The top 110 of the resting pump mount 100 may be configured such that a user's arms and head can rest on the top of the resting pump mount.

In certain embodiments, the resting pump mount 100 may have at least one divot 135. The at least one divot 135 may be located on the front 130 of the resting pump mount 100. The at least one divot 135 may be a corrugated portion on the front 130 of the resting pump mount 100. The at least one divot 135 may be configured such that a user's breasts be placed within the divot. The at least one divot 135 may be any size to fit a user's breasts. The at least one divot 135 may be bordered by a divot wall 137. The divot wall 137 may be configured to envelop at least the top, front, and bottom of at least one breast. The divot wall 137 may be further configured to envelop at least the top, front, bottom, and one side of at least one breast. The divot wall 137 may be additionally configured to envelop the top, front, bottom, and both sides of at least one breast. In certain embodiments, the at least one divot 135 may extend across the front 130 of the resting pump mount 100, from one side 150 to the other side. Alternatively, the at least one divot 135 may be cut out from the center of the front 130 of the resting pump mount 100, such that it does not extend to the sides of the resting pump mount.

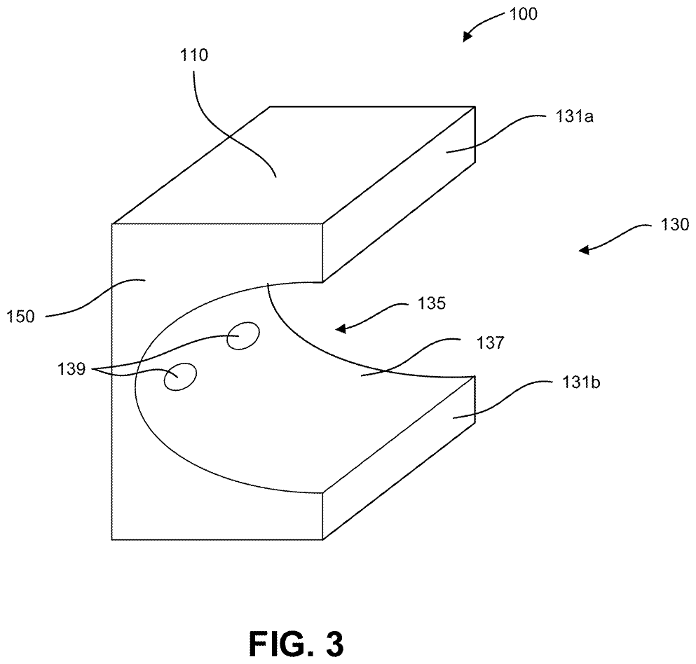

In certain embodiments, as illustrated for example in FIG. 3, the resting pump mount 100 may have at least one pump receptacle 139. The at least one pump receptacle 139 may be a feature that enhances the adaption of at least a portion of a pump to the resting pump mount 100. For example, the at least one pump receptacle 139 may include at least one pit and/or at least one hole. In these embodiments, the pump receptacle 139 may be large enough to fit at least a portion of the pump within the pit and/or hole. Alternatively, the pump receptacle 139 may be large enough to fit an entire pump within the pump receptacle. In certain embodiments, the pump receptacle 139 may include a clip, bracket, buckle, clasp, hook-and-loop attachment, and/or other fastener. In these embodiments, the pump receptacle 139 may be configured to allow at least a portion of a pump to releasably attach to the pump receptacle. In certain embodiments, a pump receptacle 139 may include at least one pit and/or at least one hole as well as at least one clip, bracket, buckle, clasp, hook-and-loop attachment, and/or other fastener. The pump receptacle 139 may include other features not mentioned here within the scope of this disclosure. A pump receptacle 139 may be located on any portion of the resting pump mount 100. In certain embodiments, the pump receptacle 139 may be located on the divot wall 137.

In certain embodiments, the resting pump mount 100 may have a body that substantially holds shape. In other words, the body will be configured to independently maintain its shape and structure. In certain embodiments, the body of the resting pump mount 100 may be at least partially compressible. For example, the resting pump mount 100 may be made of high density foam. The body of the resting pump mount 100 may be made of polystyrene. Alternatively, in certain embodiments the body of the resting pump mount 100 may be inflatable. In these embodiments, the body of the resting pump mount 100 may be inflatable with a vinyl surface. For example, the body of the resting pump mount 100 may be inflatable with a polyvinyl chloride surface. The body of the resting pump mount 100 may be made of a single material. Alternatively, the body of the resting pump mount 100 may have multiple different materials. The body of the resting pump mount 100 may have materials or additional structures that provide stiffness or stability to specific regions of the resting pump mount. For example, the resting pump mount 100 may have pleats. The pleats may be reinforced grooves in the surface of the resting pump mount. The pleats in the resting pump mount 100 may help provide structure to the resting pump mount. The divot wall 137 may have a plurality of pleats. The pleats on the divot wall 137 may be vertical to maintain a configured to maintain the divot wall 137 in a curved conformation. The body of the resting pump mount 100 may be made of any other compressible material other than the ones mentioned here within the scope of this disclosure.

The resting pump mount 100 may have at least one comfort feature. A comfort feature may be a feature that provides a soft texture on an outer surface of the resting pump mount 100. A comfort feature may be located on an outer surface where a portion of the user's skin contacts the resting pump mount 100. For example, a comfort feature may be located on the top of the resting pump mount 100. Additionally or alternatively, a comfort feature may be located on at least a portion of the divot wall 137. A comfort feature may be located on the bottom side of the resting pump mount 100. The comfort feature may be made of any soft, non-abrasive surface known in the art. For example, the comfort feature may be made of velvet, fleece, cotton, polyester, nylon, viscose, or rayon. The comfort feature may be made of any other soft material other than the ones mentioned here within the scope of this disclosure.

The resting pump mount 100 may have a variety of shapes and sizes. The resting pump mount 100 may be produced in different sizes so that it can accommodate different sized users. Alternatively, the resting pump mount 100 may be “one size fits all.” The resting pump mount 100 may also be configured to change sizes according to the size of the user. The resting pump mount 100 may be generally prism shaped. The resting pump mount 100 may have rounded edges. The resting pump mount 100 may have a generally round shape. For example, the resting pump mount 100 may be spherical or ovoid. The resting pump mount 100 may be an appropriate size and configuration to promote easy portability and storage of the resting pump mount 100. The resting pump mount 100 may also be configured to engage with one single breast.

The apparatus may include a breast pump. The breast pump may be an electric pump. The breast pump may be a manual pump. For example, the breast pump may be a Haakaa® type pump. The resting pump mount 100 may be configured to engage with a breast pump. The resting pump mount 100 may have a divot 135 appropriately sized to fit a breast pump and a user's breasts. The resting pump mount 100 may have additional divots to accommodate a breast pump. A breast pump may releasably engage with the resting pump mount 100. Alternatively, a portion of a breast pump may be fixed to the resting pump mount 100.

A user may use the apparatus during breast pumping. The user may use the apparatus while sitting upright in bed or on another surface. The user may place the resting pump mount 100 on her lap or legs such that the bottom 120 of the resting pump mount contacts her legs. The user may orient the resting pump mount 100, such that the front 130 of the resting pump mount faces her torso. The user may place her breasts within the divot 135 of the resting pump mount 100. The user may attach a breast pump to a breast while her breasts are within the divot 135. The user may use the breast pump while her breasts are inside the divot 135. While using the apparatus, the user may lean forward over the top 110 of the resting pump mount 100. The user may rest her arms and or hear on the top 110 of the resting pump mount 100. In doing so, the user may rest while using the breast pump.

The features and functions described above, as well as alternatives, may be combined into many other different systems or applications. Various alternatives, modifications, variations or improvements may be made by those skilled in the art, each of which is also intended to be encompassed by the disclosed embodiments.

1. An apparatus comprising:
a resting pump mount comprising:
a body comprising a top, bottom, front, back, first side, and second side;
wherein the front side comprises a divot sufficiently sized to fit at least one of a user's breasts,
wherein the divot is formed by a divot wall, and
wherein the body substantially holds shape and is at least partially compressible.

2. The apparatus of claim 1, wherein the divot wall is configured to envelop at least the top, front, and bottom of a breast.

3. The apparatus of any of claim 1, wherein the body comprises a high density foam.

4. The apparatus of any of claim 1, wherein the body is inflatable.

5. The apparatus of any of claim 1 further comprising at least one comfort feature, wherein the at least one comfort feature comprises a soft material.

6. The apparatus of claim 5, wherein the soft material is velvet, fleece, cotton, polyester, nylon, viscose, or rayon.

7. The apparatus of claim 5, wherein the at least one comfort feature is on at least a portion of the divot wall.

8. The apparatus of claim 5, wherein the at least one comfort feature is on at least a portion of the top and/or bottom of the body.

9. The apparatus of claim 1, further comprising a breast pump.

10. The apparatus of claim 9, wherein the breast pump is configured to releasably attached to the resting pump mount.

11. The apparatus of claim 9, wherein the breast pump is attached to the resting pump mount.

12. The apparatus of claim 1, wherein the divot extends from the first side to the second side.

13. The apparatus of claim 1, wherein the divot does not contact the first side or the second side.

14. The apparatus of claim 1 further comprising at least one pump receptacle;
wherein the at least one pump receptacle enhances adaption of at least a portion of a pump to the resting pump mount, and
wherein the at least one pump receptacle is located on the divot wall.

15. The apparatus of claim 14, wherein the pump receptacle is a pit or a hole in the divot wall.

16. The apparatus of claim 1, wherein the divot wall further comprises a plurality of pleats configured to maintain the divot wall in a curved conformation.
